(19) United States
(12) Patent Application Publication
(10) Pub. No.: US 2012/0259092 A1
(43) Pub. Date: Oct. 11, 2012

(54) NPY ANTAGONISTS

(76) Inventors: David Chatenet, Quebec (CA); Jean Claude Reubi, Wabern (CH); Jean E. F. Rivier, La Jolla, CA (US)

(21) Appl. No.: 13/444,675

(22) Filed: Apr. 11, 2012

Related U.S. Application Data
(60) Provisional application No. 61/473,960, filed on Apr. 11, 2011.

(51) Int. Cl. C07K 17/02

(52) U.S. Cl. 530/328

(57) ABSTRACT

Peptidic NPY antagonists selective for Y₁ having an organic chelator, such as DOTA, coupled thereto which are useful for diagnostic procedures and receptor-mediated radiotherapy.

### CLAIM OF PRIORITY

This patent application claims the benefit of priority, under 35 U.S.C. Section 119(e), to U.S. Provisional Patent Application Ser. No. 61/473,960, entitled “NPY ANTAGONISTS,” filed on Apr. 11, 2011 (Attorney Docket No. 3522.004PRV), which is hereby incorporated by reference herein in its entirety.

### FIELD OF THE INVENTION

This invention relates generally to short dimeric peptides that are fragment analogs of neuropeptide Y (NPY), and more particularly to such chelated dimers that are selective antagonists of receptor Y₁, that exhibit low binding affinity to receptor Y₂ and that are useful for imaging and radionuclide therapy.

### BACKGROUND OF THE INVENTION

Peptide hormone receptors, due to their over-expression on tumor cells, play an increasing role in cancer medicine; this allows specific receptor-targeted scintigraphic tumor imaging and also tumor therapy with radiolabeled peptide analogs. Somatostatin receptors were the first receptors identified for these purposes, and they have now become an integral part of the routine management of patients with gastroenteropancreatic neuroendocrine tumors. Somatostatin receptor scintigraphy (OctreoScan®) detects these tumors with extremely high sensitivity and specificity, and recent results from clinical studies performing somatostatin receptor radionuclide therapy of these tumors are very promising. The last decade has seen the development of numerous novel somatostatin agonists suitable for tumor targeting. Interestingly, however, it has recently been shown that potent somatostatin receptor antagonists, known to poorly internalize into tumor cells, can visualize tumors in vivo as well as, or even better than, the corresponding agonists. This unexpected phenomenon was found both for SSTR2- and for SSTR3-selective somatostatin analogs, and it may be due to the binding of the antagonist to a larger number of sites and/or to its lower dissociation rate. A pilot clinical trial with radiolabeled DOTA-linked SSTR2 antagonists has recently confirmed such earlier animal data.

Prompted by the success of such somatostatin receptor targeting, the over-expression of other peptide receptor families was evaluated in tumors in vivo. Promising new candidates for such an in vivo peptide receptor targeting of tumors are neuropeptide Y (NPY) receptors, based on their high expression in specific cancers, in particular breast carcinomas. In humans, at least four NPY receptor subtypes are known to exist, which are called Y₁, Y₂, Y₄ and Y₅. The natural ligands for these receptors are the peptides of the NPY family, including the neurotransmitter NPY and the two gut hormones peptide YY (PYY) and pancreatic polypeptide (PP). Through their specific interaction with the NPY receptors, these three peptides regulate a wide variety of physiologic functions, such as digestion, vasoconstriction, and reproduction and also play a key role in eating behavior. On this basis, Y₂ and Y₄ receptor agonists and Y₁ and Y₅ receptor antagonists have become potential drugs against obesity and are currently evaluated for this application. Moreover, because Y₁ and Y₂ receptors are highly over-expressed in breast cancer, Ewing sarcomas, neuroblastomas, and high grade gliomas, the use of radiolabeled Y₁ and Y₂ receptor ligands for an NPY receptor-targeted imaging and radiotherapy of these tumors has been suggested. Consequently, a Y₁-selective, daunorubicin-coupled cytotoxic NPY analog (Langer M, et al., Novel peptide conjugates for tumor-specific chemotherapy, J Med. Chem. 2001; 44:1341-48), a Y₂-selective, ^{99m}Tc-labeled radioactive NPY analog (Langer M, et al., ^{99m}Tc-Labeled Neuropeptide Y Analogues as Potential Tumor Imaging Agents, Bioconjug Chem. 2001; 12:1028-34), and more recently, a ^{99m}Tc-labeled Y₁ agonist (Khan I U, et al., Breast-cancer diagnosis by neuropeptide Y analogues: from synthesis to clinical application, Angew Chem Int Ed Engl. 2010; 49:1155-8) have been produced.

### SUMMARY OF THE INVENTION

NPY analogs coupled to a polydentate ligand or chelator suitable for radiolabeling have now been designed and developed that are useful for imaging and for radiotherapy. A DOTA-coupled, dimeric, peptidic NPY antagonist, selective for Y₁, has been synthesized, tested and found to exhibit high affinity to Y₁ and very low affinity for Y₂. Highly selective, potent peptidic NPY analogs have earlier been created which were Y₁-selective antagonists. To adopt those analogs for imaging, chelating agents, such as PADA, Dauno, Doxo and N′ His-ac were employed; however, such additions variously detracted from selectivity and/or affinity. NPY analogs have now been created which are DOTA-coupled and which retain their high selectivity and high binding affinity.

### BRIEF DESCRIPTION OF THE DRAWINGS

FIG. 1A shows the structure of a DOTA-free dimeric Y₁ selective receptor antagonist (Peptide No. 9). FIG. 1B shows one DOTA-conjugated dimeric counterpart (Peptide No. 10), and FIG. 1C shows another (Peptide No. 11).

FIG. 2 shows the results of competition experiments using the NPY Y₁ receptor expressing SK-N-MC cell line. All four tested compounds exhibit Y₁ selectivity. While hPYY () and Peptide No. 11 (♦) show high affinity displacements of ¹²⁵I-hPYY, the analogs Peptides No. 9 (▪) and No. 10 (▴) show low affinity displacements of ¹²⁵I-hPYY. Dose response curves of at least three experiments ±SEM are shown.

FIG. 3 shows the antagonist effect of Peptide No. 11 on the inhibition of the forskolin-stimulated cAMP accumulation in SK-N-MC cells. Cells were incubated for 30 min with 10 μM forskolin in the presence of [Leu³¹-Pro³⁴]-PYY (LP-PYY) at concentrations ranging between 0.01 nM and 20 μM alone () or with 10 μM forskolin in the presence of LP-PYY at concentrations ranging between 0.01 nM and 20 μM supplemented with a fixed concentration of 20 μM of the analog Peptide No. 11 (▪). Peptide No. 11 behaves like an antagonist since it shifts the dose response curve of LP-PYY to the right. Peptide No. 11 given alone has no effect on the accumulation of forskolin-stimulated cAMP (▴).

### DETAILED DESCRIPTION OF THE PREFERRED EMBODIMENTS

Neuropeptides are small peptides originating from large precursor proteins synthesized by peptidergic neurons and endocrine/paracrine cells. They hold promise for treatment of neurological, psychiatric, and endocrine disorders. Neuropeptide Y (NPY), a 36-amino acid peptide, is the most abundant neuropeptide to be identified in mammalian brain. Human NPY has the formula: H-Tyr-Pro-Ser-Lys-Pro-Asp-Asn-Pro-Gly-Glu-Asp-Ala-Pro-Ala-Glu-Asp-Met-Ala-Arg-Tyr-Tyr-Ser-Ala-Leu-Arg-His-Tyr-Ile-Asn-Leu-Ile-Thr-Arg-Gln-Arg-Tyr-NH₂ (SEQ. ID. NO. 1). Porcine and rat NPY have the same sequence except for Leu instead of Met in the 17-position. NPY forms a family (called the pancreatic polypeptide family) together with pancreatic polypeptide (PP) and peptide YY (PYY), which all consist of 36 amino acids and have a common tertiary structure, the so-called PP-fold.

Nonapeptides based upon the residues 28-36 of NPY were designed and tested which, in monomeric and dimeric form, were found to be selective for Y₁ receptor and which were potent antagonists. These analogs have variously included the substitution of proline in residue 30, an aromatic residue such as tyrosine in residue 32 and an aliphatic residue such as leucine in residue 34. Generally, dimers of two such nonapeptide analogs were found to have higher affinity than the monomer. They were dimerized through bonds between cysteine residues that were substituted in position 31. Such research was published some ten years ago in Balasubramanian et al., J Med Chem, 44, 10, 1479-1482, May 10, 2001.

Nonapeptides having some similarity to these earlier compounds have now been designed, synthesized and tested which when dimerized and coupled with DOTA continue to exhibit specificity for the Y₁ receptor and high affinity for it. As such, these compounds are considered to be excellent candidates for radiolabeling for use in imaging tumors which over-express the Y₁ receptor and for radiotherapy.

These nonapeptides are dimers of analogs of fragments of the C-terminal fragment of NPY namely, residues 28-36 with an amidated C-terminus. The C-terminal fragment of native human NPY is Ile-Asn-Leu-Ile-Thr-Arg-Gln-Arg-Tyr-NH₂. Substitutions are preferably made in positions 29, 30, 31, 32 and 34 of this nonapeptide fragment, and reference is generally made to these residues based upon the position of the particular residue in the native NPY molecule.

The dimeric peptides have the following general formula:

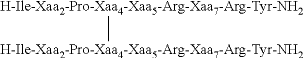

where each Xaa₂ is independently Lys, Hly, Orn, Dbu, Dpr or Asn;

each Xaa₄ independently Cys, Hey, Ncy, Glu, Asp, Lys, Orn or Dpr

each Xaa₅ is independently Trp or an aromatic L-amino acid, and

each Xaa₇ is independently Nle or Nva.

One and only one of the Xaa₂ residues is coupled to the chelator, such as DOPA, via its sidechain primary amino group.

The dimers are made by synthesizing monomer segments and then coupling two such segments to produce an 18 residue peptide dimer. The Xaa₂ residue present in the 29 position is independently Lys, homolysine (Hly), ornithine (Orn), α,γ-diaminobutyric acid (Dbu), or α,β-diaminopropionic acid (Dpr), or is the residue naturally present in NPY, namely Asn. Residue Xaa₅ is independently either Trp or an aromatic L-amino acid, such as Phe or Tyr. Residue Xaa₇ in the 34 position is an aliphatic L-amino acid, preferably an amino acid having a straight sidechain such as Nle or Nva. Residue Xaa₄ the 31 position is employed to create the dimer. Preferably Cys homocysteine (Hcy) or norcysteine (Ncy) residues are used which can be expediently linked to create an S—S disulfide bond to join the two segments into the 18 residue dimer; Cys residues are preferred. Alternatively, one of the segments can be created with a residue such as Glu or Asp having a carboxylic acid group in its sidechain, and the other segment is created with a residue having a primary amino group, preferably at the end of its sidechain, such as Lys, Orn or Dpr. In the latter instance, dimerization is effected by creating a peptidic bond between the respective sidechains.

The chelator is an organic polydentate ligand that will strongly complex with and bind radiometals, preferably one that includes at least one heterocyclic ring. Examples of such chelators include but are not limited to DTPA, DOTA, P₂, S₂-COOH, SHNH, HYNIC, NODAGA and the porphyrins; DOTA may be presently preferred. These chelators will strongly bind radiometals known to be useful for imaging and radiotherapy; examples of such radiometals include, but are not limited to ⁶⁶Ga, ⁶⁷Ga, ⁶⁸Ga, ⁸⁶Y, ⁹⁰Y, ¹¹In, ¹⁴⁹Pm, ¹⁷⁷Lu, ²⁷Mg, ⁴⁷Ca and ⁶⁴Cu.

It was surprisingly found that a chelator such as DOTA could be coupled to a dimeric peptide structure of this general type in a manner to preserve the R₁ selectivity and high binding affinity by selectively locating the DOTA moiety and by limiting DOTA-coupling to only one of the two segments or monomers of the dimer. DOTA is a known chelating agent which forms very stable complexes with a wide variety of trivalent and divalent radionuclides. In addition to limiting DOTA-coupling to one of the two segments of the dimer, it is coupled to the sidechain of a residue substituted into position 29 of the NPY fragment, as opposed to traditional coupling to the N-terminal primary amino group of such a peptide to be used for imaging purposes.

The peptides of the present invention can be synthesized by classical solution synthesis; however, they are preferably synthesized by solid phase technique as described in U.S. Pat. No. 7,019,109 dated Mar. 28, 2006, the disclosure of which is incorporated herein by reference.

Each segment of the dimeric peptides set forth hereinafter in the Example was synthesized manually on a methylbenzhydrylamine (MBHA) resin using the solid phase approach and the Boc strategy. An orthogonally protected cysteine, i.e. Boc-Cys(Acm)-OH, was used to prevent dimerization of inadequate segments. Main chain assembly was mediated by diisopropylcarbodiimide (DIC), and coupling completion was assessed by Kaiser's test. Three-fold excess of each protected amino acid was used, based on the original substitution of the MBHA resin, and Boc removal was achieved via TFA-mediated deprotection. Coupling of the DOTA moiety was performed on the monomeric fragment either at the N-terminus or at the β or ε amino group of the diaminopropionic acid (Dpr) or the Lys residue, respectively. In order to facilitate the specific linkage of the DOTA moiety, the last amino acid of the sequence was introduced as an Fmoc derivative, except for those comparative examples wherein the DOTA moiety was coupled at the N-terminus. Peptide resins were then treated with anhydrous HF in presence of anisole (5-10%, v/v) at 0° C. for 1.5 h to liberate the Cys(Acm)-crude peptides. After elimination of HF under vacuum, crude peptides were washed with peroxide-free diethyl ether and extracted with 0.1% TFA in 60% acetonitrile/water. After lyophilization, the orthogonally protected peptides were purified using preparative RP-HPLC and two successive solvent systems (A: TEAP at pH 2.25 and 0.1% TFA, B: 60% acetonitrile/water). Purified peptides were characterized by analytical RP-HPLC and MALDI-TOF-MS on a Voyager DE-STR in the reflector mode using the a-cyano-4-hydroxycinnamic acid as matrix.

Conjugation of the DOTA derivative was achieved prior to disulfide bond formation. Briefly, a solution of DOTA-NHS ester (2 eq) in DMF and N,N′-diisopropylethylamine (DIPEA) (3 eq) were added to the monomer solution in dry DMF. The mixture was stirred at room temperature and the progress of the reaction was followed by analytical RP-HPLC. After completion of the reaction and removal of the Fmoc protecting group if necessary (20% piperidine in DMF, 15 min), a preparative RP-HPLC purification was performed yielding the DOTA-conjugated monomer. Homogeneity of each fraction was assessed by analytical RP-HPLC. Removal of the Acm group was achieved through silver trifluoromethanesulfonate (100 eq/Acm) treatment of each monomer (dissolved in TFA/anisole; 99:1, 1 mg/mL) at 4° C. for 2 h, and the subsequent isolation of the peptide silver salt by centrifugation following its precipitation with ether. Dimers were obtained by treatment of two identical segments (homodimer) or two different segments (heterodimer) with aqueous 1M HCl/DMSO (1:1) overnight at room temperature resulting in the removal of the silver ions as AgCl and disulfide bond formation. Following filtration of silver chloride, dimeric peptides were once again purified and analyzed as described above.

### Example

Using this peptide synthesis regimen, 11 peptidic dimers were synthesized having the formulas set forth in the list in Table 1.

TABLE 1
Peptide Dimers Synthesized

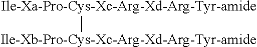

| Peptide | | | | | N-term. | | | MS^{c} | MS^{c} |
|---|---|---|---|---|---|---|---|---|---|
| number | Xa | Xb | Xc | Xd | DOTA | HPLC^{a} | CZE^{b} | calc | found |
| 1 | Asn | Asn | Tyr | Leu | No | 98% | 98% | 2390.32 | 2390.3 |
| 2 | Asn | Asn | Tyr | Leu | Yes | 97% | 96% | 3161.60 | 3162.6 |
| 3 | Asn | Asn | Trp | Nle | No | 94% | 90% | 2435.20 | 2436.5 |
| 4 | Asn | Asn | Trp | Nle | Yes | 95% | 92% | 3207.63 | 3208.8 |
| 5 | Dpr | Dpr | Trp | Nle | No | 99% | 96% | 2379.28 | 2380.1 |
| 6 | Dpr | Dpr | Trp | Nle | No | 94% | 93% | 3151.64 | 3152.4 |
| | (DOTA) | (DOTA) | | | | | | | |
| 7 | Dpr | Asn | Trp | Nle | No | 99% | 99% | 2407.27 | 2408.5 |
| 8 | Dpr | Asn | Trp | Nle | No | 98% | 97% | 2793.46 | 2794.9 |
| | (DOTA) | | | | | | | | |
| 9 | Lys | Lys | Trp | Nle | No | 88% | 84% | 2463.38 | 2464.5 |
| 10 | Lys | Lys | Trp | Nle | No | 92% | 96% | 3236.25 | 3236.8 |
| | (DOTA) | (DOTA) | | | | | | | |
| 11 | Lys | Asn | Trp | Nle | No | 83% | 83% | 2834.49 | 2835.3 |
| | (DOTA) | | | | | | | | |

^{a}Percentage purity determined by HPLC using buffer system: A = TEAP (pH 2.5) and B = 60% CH₃CN/40% A with a gradient slope of 1% B/min, at flow rate of 0.2 mL/min on a Vydac C₁₈ column (0.21 × 15 cm, 5 μm particle size, 300 Å pore size). Detection at 214 nm.
^{b}Percentage purity determined by capillary zone electrophoresis (CZE) using a Beckman P/ACE System 2050; field strength of 15 kV at 30° C. Buffer, 100 mM sodium phosphate (85:15, H2O:CH₃CN), pH 2.50, on a Agilent μSil bare fused-silica capillary (75 μm i.d. × 40 cm length). Detection at 214 nm.
^{c}MALDI mass spectral analysis (m/z). The observed m/z of the monoisotope compared with the calculated
[M + H]⁺ monoisotopic mass.

Testing of Peptides 1-11 was carried out using specific cell lines. The neuroepithelioma cell line SK-N-MC endogenously expressing the NPY Y₁ receptor was obtained from ATCC (HTB-10; LGC Standards, Teddington, Middlesex, UK). Cells were cultured at 37° C. and 5% CO₂ in MEM medium with GlutaMax I and supplemented with 10% FBS, 100 U/ml penicillin and 100 μg/ml streptomycin, 1 mM MEM sodium pyruvate, and MEM non-essential amino acids (1×). The neuroblastoma cell line SH-SY₅Y endogenously expressing the NPY Y₂ receptor, was provided by Dr. Paolo Paganetti (Novartis, Basel, Switzerland). Cells were cultured at 37° C. and 5% CO₂ in MEM/Ham's F12 with GlutaMax I and supplemented with 10% FBS, 100 U/ml penicillin and 100 μg/ml streptomycin, 1 mM MEM sodium pyruvate, and MEM non-essential amino acids (1×).

Binding affinities of the compounds were assessed using sections of cell membrane pellets of SK-N-MC for NPY Y₁ or SH-SY₅Y for NPY Y₂. Briefly, membrane pellets were prepared and stored at −80° C., and receptor autoradiography was performed on 20-μm thick cryostat sections of the membrane pellets, mounted on microscope slides and stored at −20° C. The slides were preincubated in Krebs-Ringer solution (NaCl 119 mM, KCl 3.2 mM, KH₂PO₄ 1.19 mM, MgSO₄ 1.19 mM, NaHCO₃ 25 mM, CaCl₂ 2.53 mM, D-glucose 10 mM; pH 7.4) for 60 min at room temperature. Then, they were incubated for 120 min in Krebs-Ringer solution containing 0.1% BSA, 0.05% bacitracin, and 10 000 cpm/100 μl of the ¹²⁵I-labeled human PYY alone or with increasing concentrations ranging from 0.1 nM up to 1000 nM of non-labeled hPYY, as control, or with the compounds to be tested. After the incubation, the slides were washed two times for 5 min and then rinsed four times in ice-cold preincubation solution. After drying, the slides were exposed to Kodak films Biomax MR® for 7 days. IC₅₀ values were calculated after quantification of the data using a computer-assisted image processing system. Tissue standards containing known amount of isotope, cross-calibrated to tissue-equivalent ligand concentrations were used for quantification.

Adenylate cyclase activity with respect to NPY Y₁ was determined in SK-N-MC cells using the adenylate cyclase activation flashplate assay (SMP004) from PerkinElmer. SK-N-MC cells were seeded in 96-well culture plates at 25,000 cells/well and cultured for 48 h at 37° C. and 5% CO₂. Culture medium was then removed from the wells and fresh medium (100 μL) containing 0.5 mM 3-isobutyl-1-methylxanthine (IBMX) was added to each well. Cells were incubated for 30 min at 37° C. Medium was then removed and replaced with fresh medium containing 0.5 mM IBMX, with or without 10 μM forskolin and various concentrations of the peptides to be analyzed. Cells were incubated for 30 min at 37° C. After removal of the medium, cells were lysed and cAMP accumulation was determined using the SMP004 kit from PerkinElmer.

Peptide No. 1 is a prior art dimeric Y₁ receptor antagonist that had been demonstrated to be a potent and selective NPY Y₁ antagonist. Replacement of Tyr³² by a Trp residue and introduction of a hydrophobic and bulky residue, such as norleucine (Nle), in position 34 were shown to increase Y₁ receptor affinity and selectivity as shown by testing Peptide No. 3. The DOTA-conjugated counterparts of these two peptides, i.e. Peptide Nos. 2 and 4, were then obtained through the selective addition of the chelator moiety at the N-termini of the peptide dimers; however, a significant amount of Y₁ binding affinity was lost. Position 29, i.e. Asn, was then selected as a possible alternative site for the introduction of DOTA. Introduction of the DOTA moiety was achieved through its specific attachment on the β or ε amino group, respectively, of a diaminopropionic (Dpr) or Lys residue; these two amino acids vary only by the number of carbon atoms in their sidechains. Addition of DOTA derivative was often followed by a concomitant reduction of binding affinity. We then investigated the possibility of generating asymmetric dimers bearing only one DOTA derivative and surprisingly found a significant improvement in binding affinity, selectivity, and biological activity. All peptides were purified and analyzed by RP-HPLC and CZE, and the identity was confirmed by MALDI-TOF spectrometry.

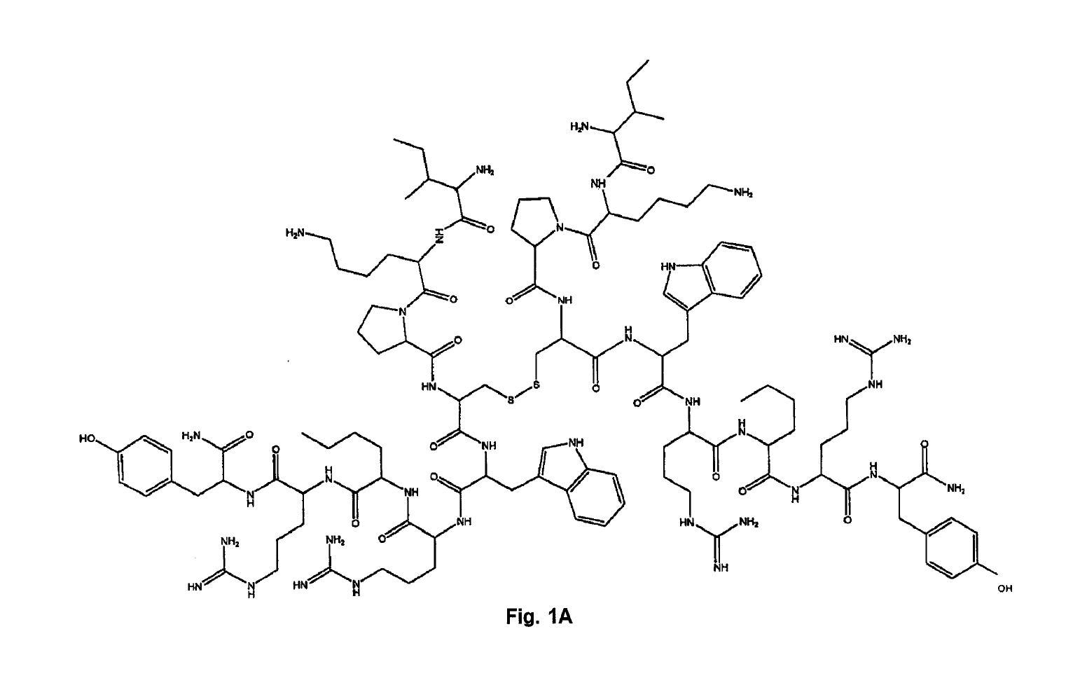

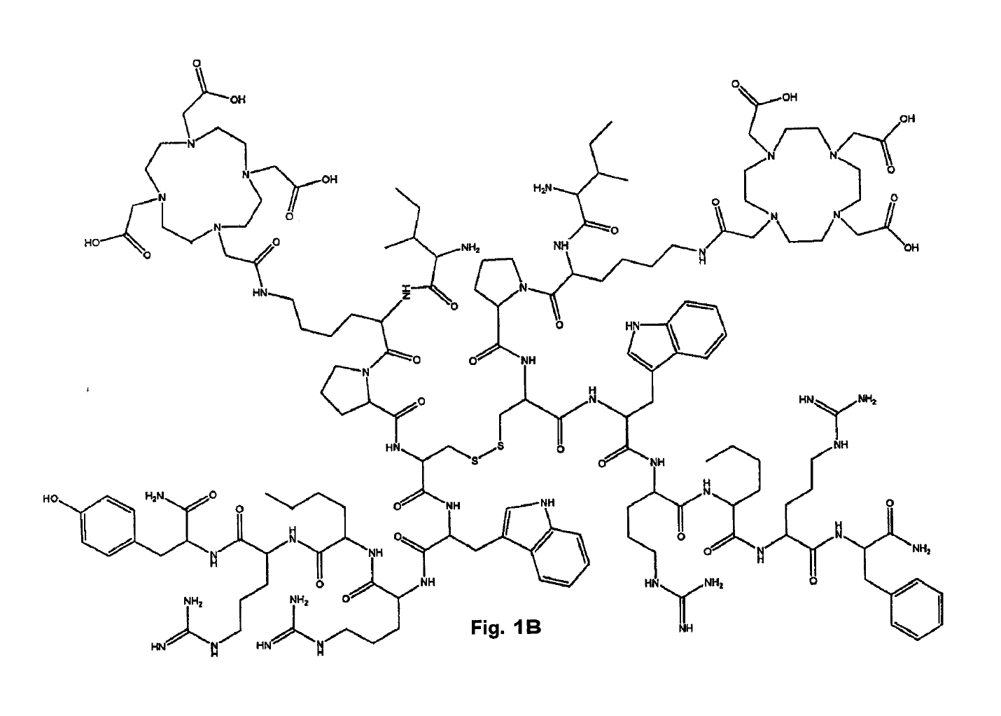

In total, 11 peptides were produced and their sequences and analytical data are listed in Table 1. Structures of (A) a DOTA-free dimeric Y₁ selective receptor antagonist (Peptide No. 9) and its DOTA-conjugated dimeric counterparts (B) Peptide No. 10 and (C) Peptide No. 11 are depicted in FIG. 1.

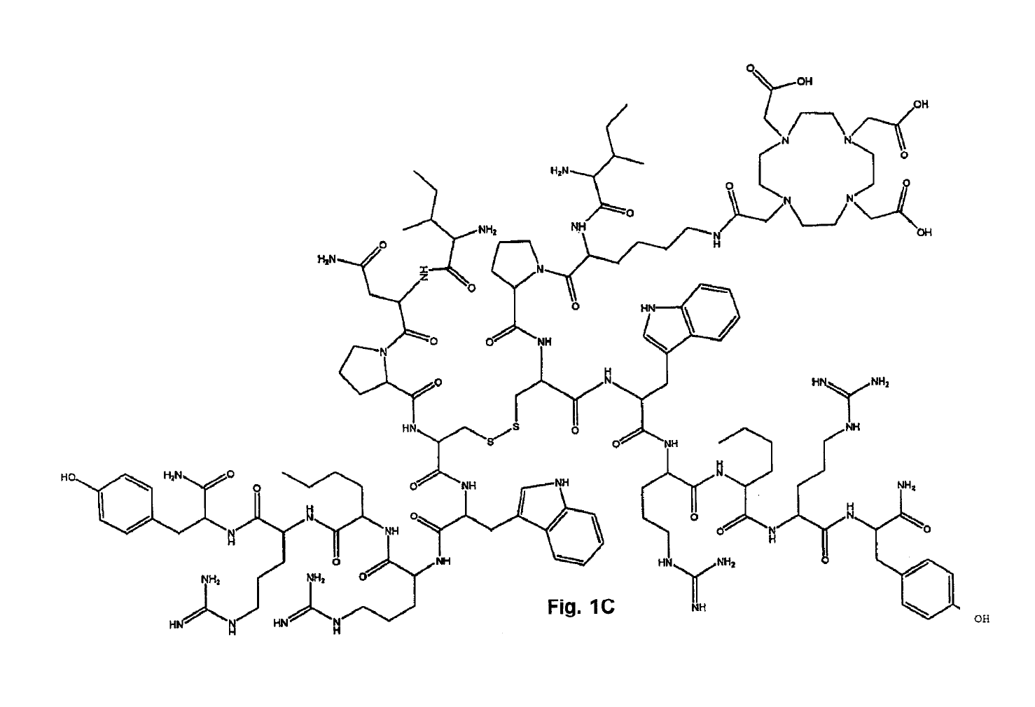

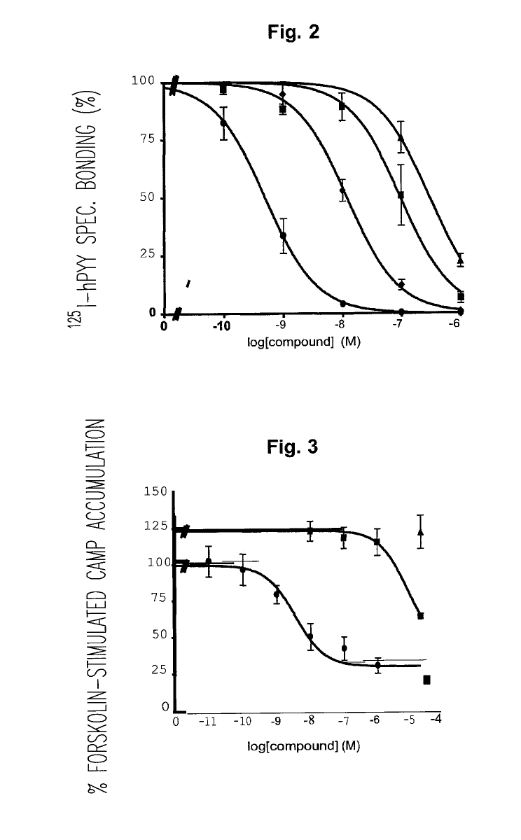

The DOTA-free and DOTA-coupled analogs listed in Table 1 were analyzed in receptor autoradiography experiments for NPY Y₁ and Y₂ receptor binding affinities on SK-N-MC cells endogenously expressing Y₁ and SH-SY5Y cells endogenously expressing Y₂, respectively. Pharmacological displacement experiment using SK-N-MC cell membrane pellet sections for Peptides Nos. 9, 10 and 11 are shown in FIG. 2. The IC₅₀ values for all tested compounds are listed in Table 2. The addition of two DOTA moieties to the homodimeric analogs decreases the Y₁ binding affinity up to 2-30 fold. However, the addition of only one DOTA to the asymmetric scaffold dimer resulted in markedly improved binding affinity, from 1 μM for Peptide No. 6 to 29 nM for Peptide No. 8. Moreover, Peptide No. 11 in which DOTA was coupled to the sidechain of a Lys residue, instead of a Dpr residue, resulted in a further enhanced binding affinity for the Y₁ receptor. None of the tested compounds showed Y₂ binding affinity.

Because the addition of a DOTA moiety can change the functional characteristics of a compound, as recently shown in the somatostatin receptor field for sst₃, compounds having a high or moderate Y₁ affinity were analyzed in an adenylate cyclase activity assay for their agonistic or antagonistic properties. The Y₁-selective agonist [Leu³¹, Pro³⁴]-hPYY (used as a positive control) efficiently inhibited forskolin-stimulated cAMP accumulation when applied at concentrations of 20 μM and 100 nM; however, all tested compounds, DOTA-free and DOTA-coupled behaved like full antagonists. Given alone at a high concentration of 20 μM, they were unable to inhibit forskolin-stimulated cAMP accumulation but they efficiently antagonized the agonistic effect of 100 nM [Leu³¹, Pro³⁴]-hPYY. FIG. 3 shows that, at 20 μM, Peptide No. 11, given together with an increasing concentration of [Leu³¹, Pro³⁴]-hPYY in the range from 10 nM up to 20 μM is able to shift the dose response curve of [Leu³¹, Pro³⁴]-hPYY to the right, indicating that this dimer efficiently antagonizes the agonist effect of [Leu³¹, Pro³⁴]-hPYY. The results of all this biological testing is included in Table 2. The conclusion is that the DOTA-conjugated compound, i.e. Peptide No. 11, with its high binding affinity and its antagonist property represents an excellent candidate for in vivo tumor targeting.

TABLE 2
Binding affinity (IC₅₀, nM) at NPY Y₁ and Y₂ receptors and
Y₁-related functional characteristics for NPY analogs.

| | Binding affinity | | Activity |
|---|---|---|---|
| | (IC₅₀ nM) | | (IC₅₀ nM) |
| | Y₁ | Y₂ | Y₁-cAMP |
| Peptide No. | (SK-N-MC) | (SH-SY5Y) | (SK-N-MC) |
| 1 | 11 ± 7.1 | >1000 | Antagonist |
| 2 | 143 ± 37 | >1000 | Antagonist |
| 3 | 9.0 ± 2.5 | >1000 | Antagonist |
| 4 | 294 ± 33 | >1000 | Antagonist |
| 5 | 143 ± 20 | >1000 | NT |
| 6 | >1000 | >1000 | NT |
| 7 | 19 ± 5.3 | >1000 | Antagonist |
| 8 | 29 ± 6.6 | >1000 | Antagonist |
| 9 | 127 ± 50 | >1000 | Antagonist |
| 10 | 283 ± 52 | >1000 | Antagonist |
| 11 | 13 ± 2.6 | >1000 | Antagonist |

The high density and incidence of Y₁ receptors in invasive and metastatic breast cancers make these neoplasms important targets for diagnosis and therapy with NPY-related drugs. Several studies have demonstrated a potential functional role of the Y₁ receptors in cancer, with preliminary experimental data suggesting that tumoral NPY receptors may be activated by long-term, non-radioactive NPY analogs and mediate tumor growth and tumoral blood supply. Radiolabeled NPY-related drugs are expected to be useful for the diagnostic localization of tumors and metastases, while radiolabeled and/or cytotoxic NPY analogs may be used for the targeted destruction of such tumors, as shown in the last decade with somatostatin radioligands. The present DOTA-coupled high affinity and Y₁-selective NPY receptor antagonists are expected to be useful tools for the diagnostic and radiotherapeutic targeting of Y₁-expressing tumors. Breast tumors with their high receptor density represent the first choice candidate tumors. Other tumor types, such as renal cell carcinomas, ovarian cancers, adrenal tumors and embryonal tumors, may also be targets of interest. The same general principles as for somatostatin receptor targeting could be applied. Advantages that should be put forward are a more favorable benefit-toxicity profile compared with conventional radio- or chemotherapy and the rarity of side effects. The targeting of tumor blood vessels alone or together with NPY receptor-expressing tumor cells may also represent an attractive strategy for therapy. Finally, since many of the NPY receptor-expressing tumors can express multiple peptide receptors concomitantly, NPY receptors may be suitable for a multireceptor targeting with a cocktail containing NPY and other therapeutic peptide analogs directed against various peptide hormone receptors. For such a multireceptor approach, good candidate tumors seem to be breast tumors targeted with NPY and bombesin analogs.

1. A peptide having the formula: 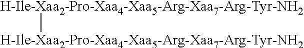
where each Xaa₂ is independently Lys, Hly, Orn, Dbu, Dpr or Asn;
each Xaa₄ is independently Cys, Hcy, Ncy, Glu, Asp, Lys, Orn or Dpr
each Xaa₅ is independently Trp or an aromatic L-amino acid, and each Xaa₇ is independently Nle or Nva; wherein only one of the Xaa₂ residues is coupled to an organic chelator via its sidechain primary amino group.

2. The peptide of claim 1 wherein one Xaa₂ is Lys.

3. The peptide of claim 2 wherein one Xaa₂ residue is Asn.

4. The peptide of claim 3 wherein both Xaa₄ residues are Cys.

5. The peptide of claim 4 wherein both Xaa₅ residues are Trp.

6. The peptide of claim 4 wherein both Xaa₇ residues are Nle.

7. The peptide of claim 1 wherein one Xaa₂ residue is Dpr.

8. The peptide of claim 7 wherein one Xaa₂ residue is Asn.

9. The peptide of claim 8 wherein both Xaa₄ residues are Cys.

10. The peptide of claim 9 wherein both Xaa₅ residues are Trp.

11. The peptide of claim 9 wherein both Xaa₇ residues are Nle.

12. The peptide of claim 1 wherein one Xaa₂ residue is Dbu.

13. The peptide of claim 12 wherein one Xaa₂ residue is Asn.

14. The peptide of claim 1 wherein one Xaa₂ residue is Lys, the other Xaa₂ residue is Asn, one Xaa₄ residue is Glu or Asp and the other Xaa₄ residue is Lys, Orn or Dpr.

15. The peptide of claim 1 wherein the chelator is selected from the group consisting of DTPA, DOTA, P₂, S₂—COOH, SHNH, HYNIC, NODAGA and the porphyrins.

16. A peptide having the formula:

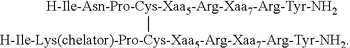

17. The peptide of claim 16 wherein the chelator is DOTA.

18. A peptide having the formula:

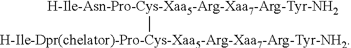

19. The peptide of claim 18 wherein the chelator is DOTA.
